(19) United States
(12) Patent Application Publication
(10) Pub. No.: US 2005/0245398 A1
(43) Pub. Date: Nov. 3, 2005

(54) Biological composition for enhancing vegetation growth

(75) Inventor: Raymond W. Stock, South Jordan, UT (US)

Correspondence Address: Michael W. Starkweather, Suite 200, 9035 South 1300East, Sandy, UT, 84094, US

(73) Assignee: Healthy Soils, Inc.

(21) Appl. No.: 11/036,422

(22) Filed: Jan. 13, 2005

Related U.S. Application Data
(60) Provisional application No. 60/566,893, filed on Apr. 29, 2004.

(51) Int. Cl. A01N 63/00; C12N 1/20

(52) U.S. Cl. 504/117; 435/253.2

(57) ABSTRACT

There is a biological composition and method of use. Included is a microorganism quantity, and a nutrient quantity which may have a supplement, a short-term nutrient and a long term nutrient. The composition may be applied to a container having water wherein the microorganisms rapidly reproduce. As the microorganisms reproduce they may be extracted. Once extracted, they may be introduced into an ecosystem, such as being injected into an irrigation system and being dispersed over crops and crop soil. There, the microorganisms may enhance vegetation growth, health, production, strength and efficiency by producing cellulase enzymes, converting nutrients to a form usable by vegetation, loosening soil, enhancing water retention in soil, and/or combating parasitic organisms.

### BACKGROUND OF THE INVENTION

This invention claims priority, under 35 U.S.C. § 119, to the U.S. Provisional Patent Application No. 60/566,893 to Raymond Stock filed on Apr. 29, 2004, which is incorporated by reference herein.

### FIELD OF THE INVENTION

The present invention relates to biological compositions, specifically to biological compositions for enhancing vegetation growth, productivity, efficiency and health by providing a quickly renewing and stable source of beneficial microorganisms.

### DESCRIPTION OF THE RELATED ART

The agriculture industry is an important component of society because it is responsible for producing food for human and animal consumption. In furtherance of this responsibility, the agricultural industry relies heavily on the soil from which crops are grown and cultivated. Over time, the repeated use of soil for growing crops can result in a diminishment of the available nutrients. As this occurs, plants can begin to show signs of inadequate nutrition by having stunted growth and/or poor health characteristics. To overcome the problems of nutrient depleted soil, farmers have been adding biological and/or nutrient containing substances to their soil for years, of which the most common is the application of animal manure. Additionally, various fertilizers have been developed for the purpose of stimulating the biological activity and nutrient level of farming soil.

While manure and fertilizers have been developed and used for many years, the results have been less than optimal. This is because some of the nutrient containing substances have to be applied several times a year, and some require applications for at least two or three years before any cost effective results are observed. Additionally, some substances are difficult to apply because of their nature to clump or otherwise not be spread evenly across the soil. Accordingly, this can cause the soil to be over-fertilized in some areas and under-fertilized in others, which results in overall poor growing conditions.

As such, improvements in nutrient rich compositions used to condition soil and the manufacturing and methods thereof continue to be sought.

### SUMMARY OF THE INVENTION

The present invention has been developed in response to the present state of the art, and in particular, in response to the problems and needs in the art that have not yet been fully solved. Accordingly, the present invention has been developed to provide a biological composition and methods of use thereof for use in ecosystems, such as conditioning soil for use by crops.

In one embodiment, there is a biological composition for enhancing vegetation growth. The biological composition may include a microorganism quantity and a nutrient quantity. The microorganism quantity may have at least a first generation of microorganisms characterized by an ability to enhance vegetation growth. The nutrient quantity may be included in an amount sufficient to sustain reproduction of the microorganism quantity for a reproduction period. The nutrient quantity may include a supplement quantity, a short-term quantity and a long-term nutrient quantity. The supplement quantity may be included in an amount sufficient to nourish the microorganism quantity for the reproduction period. The short-term nutrient quantity may be included in an amount sufficient to sustain initial reproduction of at least the first generation of microorganisms and at least a subsequent generation of microorganisms for an initial portion of the reproduction period. The long-term nutrient quantity may have a longer period of consumption by the microorganism quantity than the short-term nutrient quantity. The long-term nutrient quantity may be included in an amount sufficient to sustain continued reproduction of at least a continuing generation microorganisms and until the expiration of the reproduction period.

In another embodiment, the microorganism quantity may be selected from the group consisting of bacillus subtilis, bacillus lichenformis, bacillus cereus, bacillus megaterium, fluorescent pseudomonas, azobacter, cellulase enzyme producing bacteria, yeasts, sub-cultures thereof, and combinations thereof.

In one embodiment, the supplement quantity is selected from the group consisting of vitamins; inorganic salts, amino acids, growth media, minerals, humate, humic acids, enzymes, chelating agents, complexing agents, sequestering agents, and combinations thereof.

In another embodiment, the short-term nutrient quantity may be selected from the group consisting of hydrolyzed collagen, bone meal, blood meal, carbon skeleton molecules, sugars, carbohydrates, folvic acid, organic acid, soy protein, peptone treated biological matter, and combinations thereof.

In one embodiment, the long-term nutrient quantity may be selected from the group consisting of wheat starch, soy flour, molasses, processed or raw animal and/or plant matter, and combinations thereof.

In another embodiment, the microorganism quantity may be from about 5% to about 25% of the biological composition by volume. The nutrient quantity may be from 75% to 95% of the biological composition by volume. The supplement quantity may be from more than 0% to about 5%. The short-term nutrient quantity may be from about 50% to about 75%. The long-term nutrient quantity may be from about 15% to about 35% of the biological composition by volume.

In one embodiment, the reproduction period may be chosen from any time from about two days to about one month and the initial portion of the reproduction period is from about 10% to about 70% of the reproduction period.

In another embodiment, there may be a method of growing microorganisms for distribution into a plant eco-system, which method may include providing a container; providing a liquid mixture within the container, wherein the liquid mixture includes a water quantity, a microorganism quantity characterized by an ability to participate beneficially in a plant eco-system and a nutrient quantity; introducing oxygen into the liquid mixture; and circulating the liquid mixture.

In one embodiment, the nutrient quantity may include a short-term nutrient quantity and a long-term nutrient quantity. There may also be a supplement quantity included in the nutrient quantity.

In another embodiment, the method may further include extracting liquid mixture portions from the container at an average rate not significantly greater than an average regenerative rate of the liquid mixture.

In one embodiment, there may be a method of introducing microorganisms into a plant eco-system. This method may include providing a container; providing a liquid mixture within the container, wherein the liquid mixture includes a water quantity, a microorganism quantity characterized by the ability to participate beneficially in the plant ecosystem and a nutrient quantity; introducing oxygen into the liquid mixture; circulating the liquid mixture; extracting liquid mixture portions from the container; introducing the liquid mixture portions into irrigation water in an irrigation system configured to irrigate plants; and irrigating plants with the combined irrigation water and liquid mixture portions.

In another embodiment the liquid mixture portions may be extracted from the container at an average rate not significantly greater than an average regenerative rate of the liquid mixture. Also, the liquid mixture portions may be extracted from the container from an extraction location disposed in a lower half portion of the container.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### BRIEF DESCRIPTION OF THE DRAWINGS

In order for the advantages of the invention to be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIGS. 1 and 2 illustrate a system configured to introduce microorganisms into a plant ecosystem according to one embodiment of the invention.

### DETAILED DESCRIPTION OF THE INVENTION

For the purposes of promoting an understanding of the principles of the invention, reference will now be made to the exemplary embodiments illustrated in the drawings, and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

Reference throughout this specification to “one embodiment,” “an embodiment,” or similar language means that a particular feature, structure, or characteristic described in connection with the embodiment is included in at least one embodiment of the present invention. Thus, appearances of the phrases “one embodiment,” “an embodiment,” and similar language throughout this specification may, but do not necessarily, all refer to the same embodiment, different embodiments, or quantity parts of the same or different illustrated invention. Additionally, reference to the wording “an embodiment,” or the like, for two or more features, elements, etc. does not mean that the features are related, dissimilar, the same, etc. The use of the term “an embodiment,” or similar wording, is merely a convenient phrase to indicate optional features, which may or may not be part of the invention as claimed.

Each statement of an embodiment is to be considered independent of any other statement of an embodiment despite any use of similar or identical language characterizing each embodiment. Therefore, where one embodiment is identified as “another embodiment,” the identified embodiment is independent of any other embodiments characterized by the language “another embodiment.” The independent embodiments are considered to be able to be combined in whole or in part one with another as the claims and/or art may direct, either directly or indirectly, implicitly or explicitly.

Finally, the fact that the wording “an embodiment,” or the like, does not appear at the beginning of every sentence in the specification, such as is the practice of some practitioners, is merely a convenience for the reader's clarity. However, it is the intention of this application to incorporate by reference the phrasing “an embodiment,” and the like, at or near the beginning of every sentence herein where logically possible and appropriate.

Within this specification, including the claims, the phrase “first generation of microorganisms,” and the like, is defined to include the entire set of microorganisms initially present in the biological composition when the biological composition is first put to use as described in the specification, including those that may be reproductive descendants of others present within the biological composition at the same time.

Within this specification, including the claims, the phrase “subsequent generation of microorganisms,” and the like, is defined to include a generation of microorganisms which are reproductive descendants, whether immediate or not, of the first generation of microorganisms.

Within this specification, including the claims, the phrase “continuing generation of microorganisms,” and the like, is defined to include a generation of microorganisms which are reproductive descendants, whether immediate or not, of the subsequent generation of microorganisms.

Within this specification, including the claims, the phrase “microorganism quantity” is defined to include the microorganisms of the biological composition, including all reproductive descendants. Further it includes any and all other material which may be present with the microorganisms as they are mixed together with other materials to form the biological composition. These materials may include inert carrier materials as well as any other materials.

Within this specification, including the claims, the term “vegetation,” and the like, is defined to include all plants and other flora.

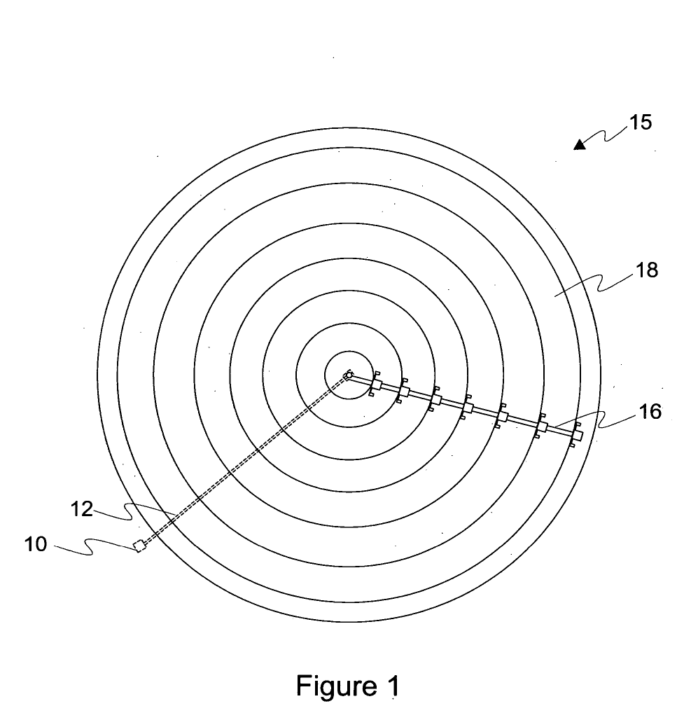

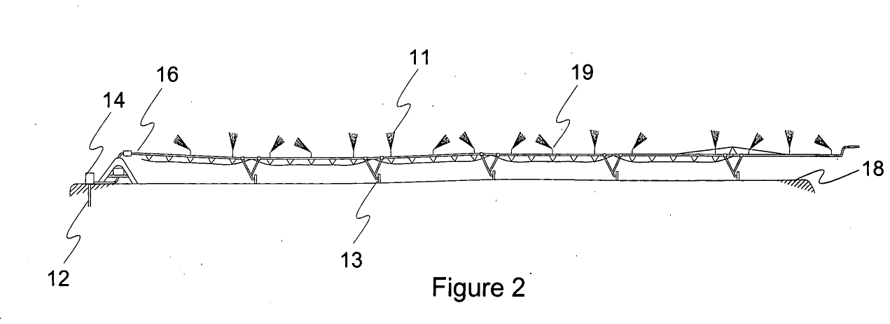

FIGS. 1 and 2 illustrate a system configured to introduce microorganisms into a plant ecosystem according to one embodiment of the invention. There is a tank, or container 10, connected to a feed pipe 12, containing irrigation water and extending underground, of an irrigation system 15 configured to irrigate a plant ecosystem 18. The irrigation system 15 further includes a junction 14 connecting the feed pipe 12 to the irrigation pipe 16. The irrigation pipe 16 includes sprinklers 19 configured to distribute fluid 11 over the plant ecosystem 18. Further, there are wheels 13 configured to permit displacement of the irrigation pipe 16 about the plant ecosystem 18.

In operation, microorganisms are generated, or regenerated, as products and further generations of a biological composition, in a liquid mixture in the container 10. The feed pipe 12 may supply water to the container 10 as needed. The liquid mixture is introduced into the feed pipe 12 at a known rate or at known portions per elapsed time. Preferably, the rate at which the liquid mixture is introduced into the feed pipe 12 does not exceed the rate at which the liquid mixture is generated by the container 10. For example, where the container generates, or regenerates, 100 gallons of liquid mixture per day, it is preferable that the rate at which the liquid mixture is introduced, or injected, into the feed pipe does not exceed 100 gallons per day, regardless of whether the rate is constant throughout a twenty-four hour period. The liquid mixture portions mix with the irrigation water as the irrigation water flows through the feed pipe 12, through the junction 14, through the irrigation pipe 16, through the sprinklers 19, which sprinklers distribute the combined fluids 11 over the plant ecosystem 18.

Microorganism generation, and/or regeneration, consumes nutrients. Further, generations of microorganisms tend to mutate, or change, from strains originally introduced. Therefore, preferably, nutrients and original strains of microorganisms are introduced into the container as needed. For example, biological compositions, preferably in pre-made packets including a nutrient quantity and a quantity of original strain(s), or microorganism quantity, are preferably placed within the container on a regular schedule, such as once a week.

In operation of one embodiment, the biological composition may provide a two stage rich source of food and other nutrients for the quantity of microorganisms. The first stage permits rapid reproduction of the microorganism. This beneficially permits the microorganism to predominate other competing microorganisms and creates a large quantity of microorganisms in a short period of time, which then may be dispersed into a plant ecosystem. The second stage facilitates maintenance of the microorganism population as portions are extracted and dispersed into a plant ecosystem. During both stages, important nutrients are supplied by the biological composition to the reproducing microorganism population.

Additionally, in operation of one embodiment, the two stage rich source of food is configured to last for a reproduction period, with the materials providing the first stage being substantially consumed before the expiration of the reproduction period. For example, the biological composition may be configured to last for a week, with the first stage configured to be substantially consumed within 48 hours. In another example, the biological composition may be configured to last 4 days, with the first stage configured to be substantially consumed within 24 hours.

One skilled in the art would know that by varying the proportions and total amounts of the materials comprising the ingredients of the two stages and the initial microorganism content of the biological composition, one may adjust the biological composition to generally conform the reproduction period to any reasonable desired period of time. Also, it is not necessary that the reproduction period be known to the manufacturer or that it be designated at all, merely that there be a period of time to which the composition relates as described herein.

Also, in operation of an embodiment, oxygen is introduced to a liquid mixture containing microorganisms in a container. This may be accomplished by injection, bubbling, interface exchange, or any other method known in the art for providing access to oxygen within a liquid. The source of oxygen may be normal air.

Still yet, in operation of one embodiment, portions of a liquid mixture containing microorganisms in a container are extracted from the container. This may be accomplished with an outlet, preferably coupled to a lower portion of the container to avoid clogging with any surface skins formed on a top surface of the liquid mixture in the container.

In addition, the nutrient quantity preferably includes a food source easily utilized by the microorganisms, or short-term nutrient, to promote rapid generation of microorganisms, in particular rapid generation of the original strain. Additionally, the nutrient quantity preferably includes a long lasting food source, or long-term nutrient, configured to nourish the microorganisms after the short-term food source depletes. Also, the nutrient quantity preferably includes a quantity of other nutrients, or supplement nutrients. The nutrient quantity may include but is not limited to vitamins, minerals, enzymes, amino acids, protein compositions, starches, fibers, carbohydrates, sugars, growth media, proteins, chelating agents, complexing agents, sequestering agents, and other materials useful in nourishing microorganisms and plants.

In particular, it is preferred that the microorganism quantity includes a microorganism(s) characterized by the ability to enhance vegetation growth, preferably in a plant dominated ecosystem such as a field of human cultivated plants. Examples of beneficial microorganisms include but are not limited to bacteria, yeasts, protozoa, actinomycites, and nematodes. It is preferred that the microorganism quantity includes a microorganism(s) characterized by the ability to produce cellulase enzymes, to convert nutrients to a form usable by plants, to loosen soil, to enhance water retention in soil, to combat parasitic organisms, and/or to otherwise enhance soil vitality, plant vitality, crop production, plant health, and/or crop production efficiency. Additionally, the microorganisms may be aerobic bacteria. The microorganism(s) may include, but is not limited to bacillus subtilis, bacillus lichenformis, bacillus cereus, bacillus megaterium, fluorescent pseudomonas, azobacter, cellulase enzyme producing bacteria, yeasts, sub-cultures thereof, and combinations thereof.

Still further, it is preferred that the microorganism(s) in the microorganism quantity be included in sufficient quantities to predominate other microorganisms which may use the biological composition to reproduce. “Other microorganisms” as used in the previous sentence may include microorganisms present in a container, in a water supply feeding into a container, strains of similar microorganisms which may have mutated from an original strain related to or identical to the microorganisms present in the microorganism quantity.

Still, in particular, it is preferred that the short-term nutrient include ingredient(s) characterized by the ability to provide a quick and ready source of nourishment for the microorganisms of the microorganism quantity. Preferably, this may include but is not limited to hydrolyzed collagen, bone meal, blood meal, carbon skeleton molecules, sugars, carbohydrates, folvic acid, organic acid, soy protein, peptone treated biological matter (such as peptone treated animal carcasses or peptone treated plant matter), other easily consumed materials and combinations thereof. Preferably, the short-term nutrient is present in the biological composition in sufficient amounts to provide for rapid reproduction of the microorganism quantity and its further generations for a reproduction period.

Further, in particular, it is preferred that the long-term nutrient include ingredients(s) characterized by the ability to provide a stable, lasting (as compared to the short-term nutrient and/or the intended period of replacement of the biological composition packets) source of nourishment for the microorganisms of the microorganism quantity. Preferably, this may include but is not limited to wheat starch, soy flour, molasses, processed or raw animal and/or plant matter, other slowly consumed proteins, fibers, starches, fats and carbohydrates and combinations thereof. Preferably, the long-term nutrient is present in the biological composition in sufficient amounts to provide for continued reproduction of the microorganism quantity and its further generations for a reproduction period after the short term nutrient quantity is substantially consumed.

Again, in particular, it is preferred that the supplement nutrient include ingredient(s) characterized by the ability to provide for the variety of nourishment needs of the microorganisms of the microorganism quantity. It is preferred that the supplement nutrient at least provide for at least one the non-energy source needs of the microorganisms of the microorganism quantity. Preferably, the ingredient(s) of the supplement nutrient may include but is not limited to food grade proteins; vitamins; inorganic salts; amino acids; growth media; minerals such as phosphate, potassium, calcium, sulfur, cobalt, copper, iron, magnesium, sodium, manganese, and zinc; humate and/or humic acids; enzymes; chelating, complexing, and/or sequestering agents with or without associated molecules; and combinations thereof. Preferably, the supplement nutrient is present in the biological composition in sufficient amounts to nourish the microorganism quantity and its further generations for a reproduction period.

Preferably, the biological composition is in a dry form wherein the biological composition may be stored for a time with the microorganisms in an inactive state. Preferably, the biological composition is a package. Preferably, the biological composition may be powder, granules or a pressed cake. In addition, the biological composition is preferably configured to aid or induce generation or regeneration of a quantity of microorganisms, preferably in a container or system configured to introduce microorganisms into a plant ecosystem. Also, preferably, the biological composition is configured to introduce or reintroduce an early generation of the microorganisms into the container or system. “Early generation” means that the microorganisms are not substantially mutated from the desired species, strains, and/or characteristics.

### EXAMPLE ONE

There is a dry microorganism amount which includes base soil bacteria, for example, the product known under the brand name Soil Response™. The product known as Soil Response™, is attributed to SafeWaze at 7411 N. Tryon Street in Charlette, N.C. 28213. The product known as Soil Response™ is a mixture of active hydrocarbon oxidizing, natural single-cell organisms, specifically for use on soil including, but is not limited to Pseudomonas Fluorescent, Azotobacter, as well as Cellulase enzymes producing bacteria. The microbes are contained in an inert preparation of a natural absorbent which has no chemical impact.

There is a dry nutrient amount which includes a micro-nutrient supplement, a long-term food source and a short term food source. The micro-nutrient supplement is manufactured by the organization having the trademark SafeWaze™ at 7411 N. Tryon Street in Charlette, N.C. 28213. It includes a blend of food grade proteins, vitamins, inorganic salts, and growth media—intended as a supplementary food supply for microorganisms. The micro-nutrient package may include, but is not limited to minerals and nutrients including phosphate, potassium, calcium, sulfur, cobalt, copper, iron, magnesium, and zinc, as well as proteins. The long-term food source is produced under the label DRI-MOL®, which is a dry molasses product manufactured by the organization having the trademark ADM found in Stanley, Wis. 54768. The ingredients include molasses, wheat Starch, calcium strearate, soy flour and lecithin. The short-term food source is hydrolyzed collagen, or HC, of type GCP-1000 which is manufactured by the organization known as Nitta Gelatin NA, INC. at 201 W. Passaic St. in Rochelle Park, N.J. 07662.

The composition amounts are detailed below, with a period of usage of seven days and a fresh clean out at the beginning of each season.

Biological Package Composition and Application Usage:

| | | Applied into Container |
|---|---|---|
| | Density | on Days 1, 7, 14, 21 |
| Soil Response TM | 6.25 ounces/cup | 0.625 cups |
| Micro-Nutrient Supplement | 6 ounces/cup | 0.125 cups |
| DRI-MOL ® | 5.5 ounces/cup | 2.5 cups |
| Hydrolyzed Collagen HC | 3.25 ounces/cup | 0.75 cups |

### EXAMPLE TWO

| | Approximate Percent by Volume |
|---|---|
| Dry Bacteria Culture | 5% |
| Yeast | 1% |
| Soybean Protein | 18.75% |
| Nutrient Mineral Mix | 18.75% |
| Humate | 18.75% |
| Granulated Sugar | 18.75% |
| Flour | 18.75% |

The nutrient mineral mix included crude protein, crude fat, salt, calcium, chlorine, magnesium, phosphorous, potassium, sodium, sulfur, cobalt, copper, iodine, iron, manganese, and zinc.

### EXAMPLE THREE

Examples of materials which are includable in embodiments include a variety of different biological and food mixes including soy protein use for animal feed supplements, mineral mixes used in animal nutrition, protein packages used in body building and dietary supplement, flours, sugar, raw molasses, yeast, various enzymes used in waste treatment, laundry soaps and the product sold under the mark Oxy Clean. Also included are various combinations of products including the product sold under the marks ViBasic, Xcite and ViPlex from the organization under the mark Vitech Industries. Further examples include the product under the mark Esp333, both liquid and dry, as well as a protein feed supplement from the organization under the mark Bio-Kinetics, soil stimulant from the organization known as Fertile Earth, along with various combinations of fertilizers.

### EXAMPLE FOUR

Using flood irrigation, a farmer raised 25% more alfalfa hay than his prior average using 6.5 gallons of a product of a biological composition as claimed dripped into the first irrigation, 15 gallons in his second irrigation, and 30 gallons in third. On two fields, the treatment so improved water retention that only two irrigations were required to achieve the same yield increase.

### EXAMPLE FIVE

Using pivot irrigation, a farmer farms 80 pivots. Most of the pivots are 125-acre pivots, with a few going up to 160 acres. One pivot is 540 acres. Due to the size of the 540 acre pivot and the time it take for one rotation of the 540 acre pivot the farmer was having problems with it drying out. This affected yields and hay quality. The farmer started applying a product of a biological composition dispersed as claimed through the pivot at a rate of 400 gallons per day. By the time of the first cutting, the hay stand was thicker and the soil was moist. Throughout the course of the year, the fields continued to see soil, yield and quality improvements.

### EXAMPLE SIX

Using pivot irrigation, a farmer had a pivot on a hillside where the high side of the field would not retain water and the alfalfa growth was always shorter. The farmer had tried other products on the field with little results. Use of the products of the biological composition commenced during the heat of the summer when water problems are at a peak. After just a few weeks of using on the products of the biological composition, at the rate of 120 gallons per day, growth on the high side of the field was as good as the rest of the field and yielding the same.

It is understood that the above-described preferred embodiments are only illustrative of the application of the principles of the present invention. The present invention may be embodied in other specific forms without departing from its spirit or essential characteristics. The described embodiment is to be considered in all respects only as illustrative and not restrictive. The scope of the invention is, therefore, indicated by the appended claim rather than by the foregoing description. All changes which come within the meaning and range of equivalency of the claims are to be embraced within their scope.

It is also understood that the words “irrigate” and “irrigation” are not limited to one or several means provide water, such as flood irrigation or pivot irrigation. Unless otherwise indicated, the terms “irrigate” and “irrigation” and similar terms are intended to indicate any means or act of providing with water or other similar fluid.

Also, although the figures illustrates a particular irrigation system and growth container, it is understood that not all details of the configuration and/or conjunction are shown. Also, it is envisioned that the invention may be adapted to couple with any system for dispersing water into a plant ecosystem, or that the invention may be an integral part of such system.

Additionally, although the figure and examples illustrate specific compositions, it is understood that the scope of the invention is to be determined by the claims and not by the specific examples.

It is also envisioned that one embodiment may include one material as a short-term nutrient while another may include that same material as a long-term nutrient, depending on the characteristics of the microorganisms in the microorganism quantity and/or the intended period of packet replacement.

One skilled in the art would know that although the invention is sometimes expressed as a mixture of quantities, it is not necessary that the quantities be distinct. For example, the same ingredient forming the short-term nutrient quantity may also supply at least some of the supplement nutrient quantity, as may be the case where a short-term nutrient quantity includes hydrolyzed collagen.

It is expected that there could be numerous variations of the design and/or configuration of embodiments of this invention. An example is that the biological composition may include additional materials beyond those disclosed. These materials may be carrier materials, inert materials, granulation materials, caking materials, or may or may not serve another function such as a beneficial function related to the packaging, shipping, storing, manufacturing, usage, and/or compliance with authority needs.

Finally, it is envisioned that the embodiments of the invention may be constructed of a variety of materials. It is envisioned that the embodiments of the invention may include materials, not herein disclosed but which may be known in the art, having properties substantially similar to those which have been disclosed.

Thus, while the present invention has been fully described above with particularity and detail in connection with what is presently deemed to be the most practical and preferred embodiment of the invention, it will be apparent to those of ordinary skill in the art that numerous modifications, including, but not limited to, variations in size, materials, shape, form, function and manner of operation, assembly and use may be made, without departing from the principles and concepts of the invention as set forth in the claims.

1. A biological composition for enhancing vegetation growth, comprising:
a microorganism quantity, having at least a first generation of microorganisms, characterized by an ability to enhance vegetation growth; and
a nutrient quantity, in an amount sufficient to sustain reproduction of the microorganism quantity for a reproduction period, including:
a supplement quantity in an amount sufficient to nourish the microorganism quantity for the reproduction period;
a short-term nutrient quantity, in an amount sufficient to sustain initial reproduction of at least the first generation of microorganisms and at least a subsequent generation of microorganisms, for an initial portion of the reproduction period; and
a long-term nutrient quantity, having a longer period of consumption by the microorganism quantity than the short-term nutrient quantity, in an amount sufficient to sustain continued reproduction of at least a continuing generation microorganisms and until the expiration of the reproduction period.

2. The biological composition of claim 1, wherein the microorganism quantity further comprises a microorganism type selected from the group consisting of bacillus subtilis, bacillus lichenformis, bacillus cereus, bacillus megaterium, fluorescent pseudomonas, azobacter, cellulase enzyme producing bacteria, yeasts, and sub-cultures thereof.

3. The biological composition of claim 1, wherein the supplement quantity is selected from the group consisting of vitamins; inorganic salts, amino acids, growth media, minerals, humates, humic acids, enzymes, chelating agents, complexing agents, and sequestering agents.

4. The biological composition of claim 1, wherein the short-term nutrient quantity is selected from the group consisting of hydrolyzed collagen, bone meal, blood meal, carbon skeleton molecules, sugars, carbohydrates, folvic acid, organic acid, soy protein, peptone treated biological matter, and combinations thereof.

5. The biological composition of claim 1, wherein the long-term nutrient quantity is selected from the group consisting of wheat starch, soy flour, molasses, raw biological matter, and processed biological matter.

6. The biological composition of claim 1, wherein the microorganism quantity is from about 1% to about 25% of the biological composition by volume, wherein the nutrient quantity is from 75% to 95% of the biological composition by volume and wherein the supplement quantity is from more than 0% to about 5%, the short-term nutrient quantity is from about 50% to about 75%, and the long-term nutrient quantity is from about 15% to about 35% of the biological composition by volume.

7. The biological composition of claim 1, wherein the microorganism quantity is characterized by an ability selected from the group consisting of an ability to produce cellulase enzymes, to convert nutrients to a form usable by plants, to loosen soil, to enhance water retention in soil, and to combat parasitic organisms.

8. A method of growing microorganisms for enhancing vegetation growth, comprising:
providing a container;
providing a liquid mixture within the container, wherein the liquid mixture includes a water quantity, a microorganism quantity characterized by an ability to enhance vegetation growth and a nutrient quantity;
introducing oxygen into the liquid mixture; and
circulating the liquid mixture.

9. The method of claim 8, wherein the microorganism quantity further comprises a microorganism type selected from the group consisting of bacillus subtilis, bacillus lichenformis, bacillus cereus, bacillus megaterium, fluorescent pseudomonas, azobacter, cellulase enzyme producing bacteria, yeasts, and sub-cultures thereof.

10. The method of claim 8, wherein the nutrient quantity includes a short-term nutrient quantity and a long-term nutrient quantity.

11. The method of claim 10, wherein the nutrient quantity further includes a supplement quantity.

12. The method of claim 11, wherein:
the supplement quantity is selected from the group consisting of vitamins; inorganic salts, amino acids, growth media, minerals, humates, humic acids, enzymes, chelating agents, complexing agents, and sequestering agents;
the short-term nutrient quantity is selected from the group consisting of hydrolyzed collagen, bone meal, blood meal, carbon skeleton molecules, sugars, carbohydrates, folvic acid, organic acid, soy protein, and peptone treated biological matter; and
the long-term nutrient quantity is selected from the group consisting of wheat starch, soy flour, molasses, raw biological matter, and processed biological matter.

13. The method of claim 11, further comprising extracting liquid mixture portions from the container at an average rate not significantly greater than an average regenerative rate of the liquid mixture.

14. A method of introducing microorganisms into a plant eco-system, comprising:
providing a container;
providing a liquid mixture within the container, wherein the liquid mixture includes a water quantity, a microorganism quantity characterized by an ability to enhance vegetation growth and a nutrient quantity;
introducing oxygen into the liquid mixture;
circulating the liquid mixture;
extracting a liquid mixture portion from the container;
introducing the liquid mixture portion into irrigation water in an irrigation system configured to irrigate plants; and
irrigating plants with the combined irrigation water and liquid mixture portion.

15. The method of claim 14, wherein the liquid mixture portion is extracted from the container at an average rate not significantly greater than an average regenerative rate of the liquid mixture.

16. The method of claim 14, wherein the liquid mixture portion is extracted from the container from an extraction location disposed in a lower half portion of the container.

17. The method of claim 14, wherein the microorganism quantity further comprises a microorganism type selected from the group consisting of bacillus subtilis, bacillus lichenformis, bacillus cereus, bacillus megaterium, fluorescent pseudomonas, azobacter, cellulase enzyme producing bacteria, yeast, and sub-cultures thereof.

18. The method of claim 14, wherein the nutrient quantity includes a short-term nutrient quantity and a long-term nutrient quantity.

19. The method of claim 19, wherein the nutrient quantity further includes a supplement quantity.

20. The method of claim 19, wherein:
the supplement quantity is selected from the group consisting of vitamins; inorganic salts, amino acids, growth media, minerals, humates, humic acids, enzymes, chelating agents, complexing agents, and sequestering agents;
the short-term nutrient quantity is selected from the group consisting of hydrolyzed collagen, bone meal, blood meal, carbon skeleton molecule, sugar, carbohydrate, folvic acid, organic acid, soy protein, and peptone treated biological matter; and
the long-term nutrient quantity is selected from the group consisting of wheat starch, soy flour, molasses, raw biological matter, and processed biological matter.
